(19) United States
(12) Patent Application Publication
(10) Pub. No.: US 2015/0057346 A1
(43) Pub. Date: Feb. 26, 2015

(54) METHODS OF MAINTAINING INTRAMUSCULAR MYOGLOBIN LEVELS, MAINTAINING MAXIMAL AEROBIC CAPACITY, AND ENHANCING THE OXIDATIVE CAPACITY OF MUSCLE IN A SUBJECT

(71) Applicant: ABBOTT LABORATORIES, ABBOTT PARK, IL (US)

(72) Inventors: BENJAMIN MEADOR, Monticello, IL (US); SUZETTE PEREIRA, Westerville, OH (US); NEILE EDENS, Columbus, OH (US); SUSAN GAWEL, LaGrange Park, IL (US); RAJ CHANDRAN, Evanston, IL (US); GERARD DAVIS, Wauconda, IL (US); MENGHUA LUO, New Albany, OH (US)

(73) Assignee: ABBOTT LABORATORIES, ABBOTT PARK, IL (US)

(21) Appl. No.: 14/465,563

(22) Filed: Aug. 21, 2014

Related U.S. Application Data
(60) Provisional application No. 61/869,474, filed on Aug. 23, 2013.

(51) Int. Cl. A61K 31/22

(52) U.S. Cl. CPC A61K 31/22
USPC 514/546

(57) ABSTRACT

Methods of maintaining intramuscular myoglobin levels, maintaining maximal aerobic capacity, and enhancing the oxidative capacity of muscle in a subject in need thereof are provided. Generally, the methods include administering an effective amount of β-hydroxy-β-methylbutyrate to the subject. The β-hydroxy-β-methylbutyrate may be administered as part of a nutritional composition.

### FIELD

The general inventive concepts relate to methods of improving muscle health, and more particularly to the use of an effective amount of β-hydroxy-β-methylbutyrate to maintain intramuscular myoglobin levels, to maintain maximal aerobic capacity, to enhance the oxidative capacity of muscle, or combinations thereof.

### BACKGROUND

It is generally known that aging, prolonged periods of physical inactivity, as well as chronic conditions can lead to declines in muscle health. A decline in muscle health can have a number of adverse effects on an individual including, but not limited to, general weakness, fatigue, a lessening of joint mobility, a reduction in physical activities, vulnerability to falls, and a general decline in functional status.

### SUMMARY

The general inventive concepts relate to methods of maintaining intramuscular myoglobin levels, maintaining maximal aerobic capacity, and enhancing the oxidative capacity of muscle in a subject in need thereof. By way of example to illustrate various aspects of the general inventive concepts, several exemplary embodiments of methods are provided herein.

In one exemplary embodiment, a method of maintaining intramuscular myoglobin levels in a subject in need thereof is provided. The method comprises administering an effective amount of β-hydroxy-β-methylbutyrate to the subject in need thereof. In one exemplary embodiment, the β-hydroxy-β-methylbutyrate is administered orally. In one exemplary embodiment, the β-hydroxy-β-methylbutyrate is administered as part of a nutritional composition. In one exemplary embodiment, the nutritional composition comprises at least one source of protein, at least one source of carbohydrate, and at least one source of fat.

In one exemplary embodiment, a method of maintaining the maximal aerobic capacity of a subject in need thereof is provided. The method comprises administering an effective amount of β-hydroxy-β-methylbutyrate to the subject in need thereof. Administration of the β-hydroxy-β-methylbutyrate increases intramuscular myoglobin levels, and thereby maintains the maximal aerobic capacity of the subject. In one exemplary embodiment, the β-hydroxy-β-methylbutyrate is administered orally. In one exemplary embodiment, the β-hydroxy-β-methylbutyrate is administered as part of a nutritional composition. In one exemplary embodiment, the nutritional composition comprises at least one source of protein, at least one source of carbohydrate, and at least one source of fat.

In one exemplary embodiment, a method of enhancing the oxidative capacity of muscle in a subject in need thereof is provided. The method comprises administering an effective amount of β-hydroxy-β-methylbutyrate to the subject in need thereof. Administration of the β-hydroxy-β-methylbutyrate increases intramuscular myoglobin levels, and thereby enhances the oxidative capacity of muscle in the subject. In one exemplary embodiment, the β-hydroxy-β-methylbutyrate is administered orally. In one exemplary embodiment, the β-hydroxy-β-methylbutyrate is administered as part of a nutritional composition. In one exemplary embodiment, the nutritional composition comprises at least one source of protein, at least one source of carbohydrate, and at least one source of fat.

In one exemplary embodiment, the subject in need thereof is a human. In one exemplary embodiment, the subject in need thereof is an elderly human. In one exemplary embodiment, the subject in need thereof is hospitalized, immobilized, physically inactive, or on bed rest. In one exemplary embodiment, the subject in need thereof has a condition selected from joint disease, chronic obstructive pulmonary disorder, congestive heart failure, emphysema, and asthma.

### DETAILED DESCRIPTION

While the general inventive concepts are susceptible of embodiment in many different forms, described herein in detail are specific embodiments thereof with the understanding that the present disclosure is to be considered as an exemplification of the principles of the general inventive concepts. Accordingly, the general inventive concepts are not intended to be limited to the specific embodiments illustrated and described herein.

The general inventive concepts described herein generally relate to methods of maintaining intramuscular myoglobin levels, maintaining maximal aerobic capacity, and enhancing the oxidative capacity of muscle in a subject in need thereof. The exemplary methods generally include administering an effective amount of β-hydroxy-β-methylbutyrate (HMB) to maintain or increase intramuscular myoglobin levels. Accordingly, the exemplary methods are useful for maintaining the maximal aerobic capacity of a subject in need thereof, enhancing the oxidative capacity of muscle in a subject in need thereof, or both.

The terminology as set forth herein is for description of the exemplary embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

The term “nutritional composition,” as used herein, unless otherwise specified, refers to a nutritional product in various forms including, but not limited to, liquids, solids, powders, semi-solids, semi-liquids, nutritional supplements, and other nutritional products known in the art. A nutritional composition in powder form may often be reconstituted to form a nutritional composition in liquid form. In certain exemplary embodiments, the nutritional composition comprises at least one source of protein, at least one source of carbohydrate, at least one source of fat, or combinations thereof. The nutritional compositions disclosed herein are generally suitable for oral consumption by a human. The terms “nutritional composition” and “nutritional product” may be used interchangeably.

The term “subject,” as used herein, unless otherwise specified, refers to a mammal, including companion animals, livestock, laboratory animals, working animals, sport animals, and humans. In certain exemplary embodiments, the subject is a human.

The term “subject in need thereof,” as used herein, unless otherwise specified, refers to a subject exhibiting or at risk of a decline in muscle health. A decline in muscle health may be characterized by reduced intramuscular myoglobin levels, reduced maximal aerobic capacity, reduced oxidative capacity of muscle, or combinations thereof. In certain exemplary embodiments, the subject in need thereof is an elderly human, an inactive elderly human, a diseased elderly human, or an elderly human that is both inactive and diseased. In certain exemplary embodiments, the subject in need thereof is a human undergoing a temporary or permanent period of physical inactivity, due to disability, temporary injury, bed rest, hospitalization, or recovery from surgery. In certain exemplary embodiments, the subject in need thereof is a human undergoing rehabilitation (i.e., physical rehabilitation) due to disease, injury, surgery, hospital admission, or combinations thereof. In certain exemplary embodiments, the subject in need thereof is a human having a disease condition selected from joint disease, chronic obstructive pulmonary disorder, congestive heart failure, emphysema, cachexia, diabetes, sarcopenia, end stage renal disease, and asthma.

The term “elderly,” as used herein, refers to a subject of at least 45 years of age, including at least 50 years of age, at least 55 years of age, at least 60 years of age, at least 65 years of age, at least 70 years of age, at least 75 years of age, and including at least 80 years of age or greater. The term “elderly” also includes subjects having an age between 45 years and 100 years, and subjects having an age between 55 years and 80 years.

The terms “administer,” “administering,” “administered,” and “administration” as used herein, unless otherwise specified, should be understood to include providing an agent (e.g., β-hydroxy-β-methylbutyrate, a nutritional composition) to a subject, the act of consuming the agent by the subject, and combinations thereof. In addition, it should be understood that the exemplary methods described herein, which involve administering, may be practiced with or without doctor supervision or other medical direction.

The term “effective amount” as used herein, unless otherwise specified, refers to a sufficient amount of β-hydroxy-β-methylbutyrate to maintain intramuscular myoglobin levels or to increase intramuscular myoglobin levels, and to exhibit a therapeutic effect (e.g., maintain maximal aerobic capacity, enhance the oxidative capacity of muscle). The exact amount of β-hydroxy-β-methylbutyrate required will often/typically vary from subject to subject, depending on the species, age, weight, lifestyle and general condition of the particular subject.

The terms “nutritional liquid” and “liquid,” as used herein with respect to a nutritional composition, refer to nutritional compositions in ready-to-drink liquid form, concentrated liquid form, and nutritional compositions made by reconstituting a powder with water or another aqueous liquid prior to administration. A nutritional composition in liquid form may be formulated as a suspension, an emulsion, a solution, and so forth.

The terms “nutritional powder” and “reconstitutable powder,” as used herein with respect to a nutritional composition, refers to nutritional compositions in flowable or scoopable form that can be reconstituted with water or another aqueous liquid prior to administration and includes spray dried, dry-mixed, or dry-blended powders.

The term “semi-solid,” as used herein with respect to a nutritional composition, refers to nutritional compositions that are intermediate in properties, such as rigidity, between solids and liquids. Some semi-solid examples include puddings, yogurts, gels, gelatins, and doughs.

The term “semi-liquid,” as used herein with respect to a nutritional composition, refers to nutritional compositions that are intermediate in properties, such as flow properties, between liquids and solids. Some semi-liquid examples include thick shakes, liquid yogurts, and liquid gels.

The term “serving” as used herein, unless otherwise specified, is intended to be construed as any amount which is intended to be consumed by a subject in one sitting or within one hour or less.

The term “muscle” as used herein, unless otherwise specified, refers to skeletal muscle and other non-skeletal, striated muscles such as diaphragm, extraocular muscle, and so forth.

The term “intramuscular” as used herein, unless otherwise specified, refers to all cellular parts that comprise a skeletal muscle group including, but not limited to, myofibers, myoblasts, satellite cells, neurons, endothelial cells, pericytes, monocytes, macrophages, adipocytes, and fibroblasts.

The term “providing” as used herein within the context of providing a nutritional composition or an amount of an active ingredient (e.g., β-hydroxy-β-methylbutyrate) to a subject according to a certain regimen or schedule, should be understood to reflect a subject who has been instructed to be administered a nutritional composition or an amount of active ingredient, and who actually is administered the nutritional composition or amount of active ingredient for at least 70% of the days during the desired period of the regimen or schedule. In other embodiments, providing a nutritional composition or an amount of an active ingredient (e.g., β-hydroxy-β-methylbutyrate) to a subject according to a certain regimen or schedule, should be understood to reflect a subject who has been instructed to be administered the active ingredients, and who actually is administered the nutritional composition or amount of active ingredient for at least 90% of the days during the desired period of the regimen or schedule.

All percentages, parts, and ratios, as used herein, are by weight of the total composition, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various exemplary nutritional compositions described herein may also be substantially free of any optional or selected essential ingredient or feature described herein, provided that the nutritional composition still contains all of the required ingredients or features as described herein. In this context, and unless otherwise specified, the term “substantially free” means that the selected nutritional composition contains less than a functional amount of the optional or selected ingredient, typically less than 0.5%, including less than 0.25%, including less than 0.1%, and also including zero percent, by weight, of such optional or selected ingredient.

The exemplary methods may use and the exemplary nutritional compositions may comprise, consist of, or consist essentially of the elements of the nutritional compositions as described herein, as well as any additional or optional element described herein or otherwise known (now or in the future) to be useful in certain exemplary applications.

Methods of maintaining intramuscular myoglobin levels, maintaining maximal aerobic capacity, and enhancing the oxidative capacity of muscle in a subject in need thereof are provided herein. The exemplary methods described herein include administering an effective amount of β-hydroxy-β-methylbutyrate to a subject in need thereof. In one exemplary embodiment, the β-hydroxy-β-methylbutyrate is administered to the subject in need thereof as part of a nutritional composition.

Myoglobin is a small, monomeric protein that is present in the muscle tissue of vertebrates in general and almost all mammals. Myoglobin serves as a reserve supply of oxygen and facilitates the transport of oxygen within muscle tissue. Accordingly, intramuscular myoglobin levels are important for facilitating oxygen transport and oxidative metabolism in the muscle. The exemplary methods described herein are useful for maintaining or increasing intramuscular myoglobin levels.

In one exemplary embodiment, a method of maintaining intramuscular myoglobin levels in a subject in need thereof is provided. The method comprises administering an effective amount of β-hydroxy-β-methylbutyrate to the subject in need thereof.

In one exemplary embodiment, a method of maintaining the maximal aerobic capacity of a subject in need thereof is provided. The method comprises administering an effective amount of β-hydroxy-β-methylbutyrate to the subject in need thereof. Administration of the β-hydroxy-β-methylbutyrate increases intramuscular myoglobin levels, and thereby maintains the maximal aerobic capacity of the subject.

In one exemplary embodiment, a method of enhancing the oxidative capacity of muscle in a subject in need thereof is provided. The method comprises administering an effective amount of β-hydroxy-β-methylbutyrate to the subject in need thereof. Administration of the β-hydroxy-β-methylbutyrate increases intramuscular myoglobin levels, and thereby enhances the oxidative capacity of muscle in the subject.

As mentioned above, the exemplary methods described herein comprise administering an effective amount of β-hydroxy-β-methylbutyrate to the subject in need thereof. β-hydroxy-β-methylbutyrate (also referred to as β-hydroxy-β-methylbutyric acid or HMB) is a metabolite of the essential amino acid leucine formed by transamination to α-ketoisocaproate (KIC) in muscle followed by oxidation of the KIC in the cytosol of the liver to form HMB. A variety of suitable forms of HMB may be used in the exemplary methods described herein. For example, in one exemplary embodiment, the HMB is administered in free acid form. In one exemplary embodiment, the HMB is administered as a salt of HMB. In one exemplary embodiment, the HMB is administered as an ester of HMB (e.g., methyl ester, ethyl ester). In one exemplary embodiment, the HMB is administered as a lactone of HMB (e.g., isovaleryl lactone). In one exemplary embodiment, the HMB is in the form of a non-toxic, edible salt. In one exemplary embodiment, the HMB salt is water-soluble or becomes water-soluble in the stomach or intestines of a subject. In one exemplary embodiment, the HMB salt is selected from a sodium salt, a potassium salt, a magnesium salt, a chromium salt, and a calcium salt. However, in certain other embodiments, other non-toxic salts, such as other alkali metal or alkaline earth metal salts of HMB, may be used.

In one exemplary embodiment, a suitable form of HMB that may be utilized is the calcium salt of HMB, also designated as Ca-HMB, which is most typically the monohydrate calcium salt. Calcium HMB monohydrate is commercially available from Technical Sourcing International (TSI) of Salt Lake City, Utah. When referring to particular amounts of HMB herein, the amounts are based on the assumption that the HMB is being provided as Ca-HMB, unless specifically indicated otherwise.

In one exemplary embodiment, the HMB is administered orally. The terms “administered orally” and “oral administration,” as used herein, include any form of administration in which the HMB is introduced into the subject's digestive system, including the stomach and small intestine. For example, oral administration includes nasogastric intubation, in which a tube is run through the nose to the stomach of the subject to administer food or drugs.

As discussed above, the effective amount of HMB administered to the subject may vary widely. In one exemplary embodiment, about 0.1 g/day to about 10 g/day of HMB is administered to the subject in need thereof. In one exemplary embodiment, a subject may benefit from the administration of at least 1 g/day of HMB, at least 2 g/day of HMB, about 3 g/day to about 10 g/day of HMB, or about 4 g/day to about 8 g/day of HMB. Alternatively, in one exemplary embodiment, the total daily dose of HMB administered to the subject may be between about 20 mg/kg body weight per day and about 40 mg/kg body weight per day.

In one exemplary embodiment, the HMB may be administered alone, without a carrier. For example, the HMB may be dissolved in water and consumed by the subject. Alternatively, the HMB may be sprinkled on food, dissolved in coffee, and so forth. In one exemplary embodiment, the HMB may be incorporated into pills, capsules, rapidly dissolved tablets, lozenges, and so forth. Methods for preparing such dosage forms are well known in the art. The reader's attention is directed to the most recent edition of Remington's Pharmaceutical Sciences for guidance on how to prepare such dosage forms. In one exemplary embodiment, the HMB may be combined with additional supplements such as amino acids.

Although the HMB may be administered as a single entity without a carrier, the HMB may also be incorporated into food products and consumed by the subject during their meals or snacks. In one exemplary embodiment, the HMB may be administered as part of a nutritional composition. In other words, the HMB may be incorporated into a nutritional composition, which may then be administered to the subject.

In one exemplary embodiment, the nutritional composition comprises about 0.1 grams to about 10 grams of HMB per serving of the nutritional composition. In certain exemplary embodiments, the nutritional composition comprises about 0.5 grams to about 5 grams of HMB per serving of the nutritional composition, about 0.75 grams to about 4 grams of HMB per serving of the nutritional composition, or about 1.5 to about 3 grams of HMB per serving of the nutritional composition. In one exemplary embodiment, about 0.1 g/day to about 10 g/day of HMB is administered to the subject in need thereof as part of a nutritional composition.

In one exemplary embodiment, the nutritional composition comprises at least one source of protein. In one exemplary embodiment, the at least one source of protein is present in an amount sufficient to provide between about 5 grams and about 25 grams, between about 10 grams and about 20 grams, or between about 10 grams and about 15 grams of protein per serving. Alternatively, the amount of protein present in the nutritional composition may be expressed in terms of grams of protein per liter (g/L). In such instances, the nutritional compositions comprise a source of protein in an amount sufficient to provide between about 30 grams and about 160 grams of protein per liter of the nutritional composition, between about 35 grams and about 110 grams of protein per liter of the nutritional composition, or between about 40 grams and about 60 grams of protein per liter of the nutritional composition. Alternatively, the amount of protein may be expressed in terms of the percent of total calories of the nutritional composition represented by the protein. In such instances, the nutritional composition may comprise a source of protein in an amount sufficient to provide between about 15% and about 75% or between about 15% and about 25% of the total calories of the nutritional composition.

Various sources of protein, including one source or more than one source, may be utilized in nutritional compositions according to the exemplary embodiments. Proteins suitable for use in the nutritional compositions include, but are not limited to, hydrolyzed, partially hydrolyzed, or non-hydrolyzed (intact) proteins or protein sources, and can be derived from any known or otherwise suitable source such as milk (e.g., casein, whey), animal (e.g., meat, fish), cereal (e.g., rice, corn), vegetable (e.g., soy, pea, potato), or combinations thereof.

Non-limiting examples of the source of protein include whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, skim milk, low fat milk, nonfat dry milk, skim milk powder, condensed skim milk, soy protein concentrates, soy protein isolates, soy protein hydrolysates, pea protein concentrates, pea protein isolates, pea protein hydrolysates, collagen proteins, collagen protein isolates, potato protein, rice protein, corn protein, wheat protein, sunflower protein, chickpea protein, quinoa protein, insect proteins, earthworm proteins, and combinations thereof.

In one exemplary embodiment, the nutritional composition comprises at least one source of carbohydrate. In one exemplary embodiment, the at least one source of carbohydrate is present in an amount sufficient to provide between about 10 grams and about 100 grams of carbohydrates per serving. The carbohydrates may be from one source or a variety of sources. In one exemplary embodiment, the nutritional composition comprises at least one source of carbohydrate in an amount sufficient to provide between about 20 grams and about 60 grams, between about 30 grams and about 60 grams, between about 40 grams and about 60 grams, or between about 50 grams and about 60 grams of carbohydrates per serving. Alternatively, the amount of carbohydrates present in the nutritional composition may be expressed in terms of grams of carbohydrates per liter (g/L). In such instances, the nutritional composition comprises a source of carbohydrate in an amount sufficient to provide between about 125 grams and about 255 grams of carbohydrates per liter of the nutritional composition, or between about 150 grams and about 230 grams of carbohydrates per liter of the nutritional composition. Alternatively, the amount of carbohydrates may be expressed in terms of the percent of total calories of the nutritional composition represented by the carbohydrates. In such instances, the nutritional composition comprises a source of carbohydrate in an amount sufficient to provide between about 30% and about 75% or between about 50% and about 65% of the total calories of the nutritional composition.

The at least one source of carbohydrate may be simple, complex, or variations or combinations thereof. A wide variety of sources of carbohydrates may be used so long as the source is suitable for use in oral nutritional compositions and is otherwise compatible with the other selected ingredients or features of the nutritional composition. Non-limiting examples of a source of carbohydrate which may be suitable for use in the exemplary nutritional compositions described herein include maltodextrin, hydrolyzed or modified starch or cornstarch, glucose polymers, corn syrup, corn syrup solids, rice-derived carbohydrates, sucrose, glucose, fructose, lactose, high fructose corn syrup, honey, sugar alcohols (e.g., maltitol, erythritol, sorbitol), isomaltulose, sucromalt, pullulan, potato starch, and other slowly-digested carbohydrates, dietary fibers including, but not limited to, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, inulin, fructooligosaccharides, low and high methoxy pectin, cereal beta-glucans (e.g., oat beta-glucan, barley beta-glucan), carrageenan and psyllium, Fibersol™, other resistant starches, and combinations thereof.

In one exemplary embodiment, the nutritional composition comprises at least one source of fat. In one exemplary embodiment, the at least one source of fat is present in an amount sufficient to provide between about 0.001 grams and about 20 grams, between about 0.5 grams and about 18 grams, or between about 1 gram and about 15 grams of fat per serving. Alternatively, the amount of fat present in the exemplary nutritional compositions may be expressed in terms of grams of fat per liter (g/L). In such instances, the nutritional composition comprises a source of fat in an amount sufficient to provide between about 4 grams and about 85 grams of fat per liter, or between about 10 grams and about 50 grams of fat per liter of the nutritional composition. Alternatively, the amount of fat may be expressed in terms of the percent of total calories of the nutritional composition represented by the fat. In such instances, the nutritional composition comprises a source of fat in an amount sufficient to provide between about 10% and about 75% or between about 20% and about 40% of the calories in the nutritional composition.

Non-limiting examples of fats suitable for use in the exemplary nutritional compositions include canola oil, corn oil, coconut oil, fractionated coconut oil, soy oil, olive oil, safflower oil, high GLA (gamma-linolenic acid) safflower oil, high oleic safflower oil, MCT (medium chain triglycerides) oil, sunflower oil, high oleic sunflower oil, palm and palm kernel oils, palm olein, marine oils, cottonseed oils, algal and fungal derived oils, and combinations thereof.

In one exemplary embodiment, the nutritional composition comprises at least one source of protein, at least one source of carbohydrate, and at least one source of fat. In one exemplary embodiment, the nutritional composition comprises at least one source of protein and at least one source of carbohydrate. In one exemplary embodiment, the nutritional composition comprises at least one source of protein and at least one source of fat. In one exemplary embodiment, the nutritional composition may comprise at least one source of carbohydrate and at least one source of fat. The at least one source of protein may be selected from one or more than one of the previously listed sources of protein. The at least one source of carbohydrate may be selected from one or more than one of the previously listed sources of carbohydrate. The at least one source of fat may be selected from one or more than one of the previously listed sources of fat.

In one exemplary embodiment, the nutritional composition comprises at least one vitamin and at least one mineral. For example, in certain exemplary embodiments, the nutritional composition comprises vitamins and minerals that have antioxidant properties such as vitamin E, Vitamin C, selenium, molybdenum, and combinations thereof. In certain exemplary embodiments, the nutritional composition comprises vitamin D, including vitamin D₂ and vitamin D₃, which promotes intestinal absorption of calcium and phosphate. In certain exemplary embodiments, the nutritional composition comprises between 125 IUs and 200 IUs or between 150 IUs and 175 IUs of vitamin D (1 IU of vitamin D is equivalent to 0.025 micrograms of vitamin D) per serving.

In certain exemplary embodiments, the nutritional composition may include other vitamins and related nutrients, non-limiting examples of which include vitamin A, vitamin A palmitate, vitamin E acetate, vitamin C palmitate (ascorbyl palmitate), vitamin K, thiamine, riboflavin, pyridoxine, vitamin B₁₂, carotenoids (e.g., beta-carotene, zeaxanthin, lutein, lycopene), niacin, folic acid, pantothenic acid, biotin, choline, inositol, salts and derivatives thereof, and combinations thereof. In certain exemplary embodiments, the nutritional composition may comprise a wide variety of additional minerals, non-limiting examples of which include calcium, potassium, iodine, phosphorus, magnesium, iron, zinc, manganese, copper, sodium, chromium, chloride, and combinations thereof.

The exemplary nutritional compositions can provide up to about 500 kcal of energy per serving, including between about 240 kcal and about 500 kcal, between about 275 kcal and about 450 kcal, between about 300 kcal and about 400 kcal, between about 325 kcal and about 375 kcal, or between about 325 kcal and about 350 kcal per serving.

In one exemplary embodiment, the nutritional composition may comprise other optional ingredients that may modify the physical, chemical, aesthetic, or processing characteristics of the nutritional composition, or serve as additional nutritional components. Many such optional ingredients are known or otherwise suitable for use in medical food or other nutritional products and may also be used in the nutritional compositions described herein, provided that such optional ingredients are safe for oral administration and are compatible with the essential and other ingredients in the selected product form.

In one exemplary embodiment, the nutritional composition may comprise at least one sweetening agent. In certain exemplary embodiments, the at least one sweetening agent is a sugar alcohol such as maltitol, erythritol, sorbitol, xylitol, mannitol, isomalt, and lactitol, or at least one artificial or high potency sweetener such as acesulfame K, aspartame, sucralose, saccharin, stevia, and tagatose, and combinations thereof. The sweetening agents, especially as a combination of a sugar alcohol and an artificial sweetener, can be useful in formulating liquid nutritional compositions having a desirable favor profile. These sweetener combinations can also be effective in masking undesirable flavors, for example, as sometimes associated with the addition of vegetable proteins to a liquid nutritional composition.

In one exemplary embodiment, the nutritional composition may comprise a flowing agent or anti-caking agent to retard clumping or caking of a nutritional powder embodiment over time and to make the nutritional powder flow easily from its container. Any flowing or anti-caking agents that are known or otherwise suitable for use in a nutritional powder are suitable for use herein, non-limiting examples of which include tricalcium phosphate, silicates, and combinations thereof. The concentration of the flowing agent or anti-caking agent will often vary depending upon the product form, the other selected ingredients, the desired flow properties, and so forth.

In one exemplary embodiment, the nutritional composition may comprise a stabilizer. Any stabilizer that is known or otherwise suitable for use in a nutritional composition may also be suitable for use herein, non-limiting examples of which include gums such as xanthan gum and locust bean gum.

In certain exemplary embodiments, the nutritional composition may include one or more masking agents to reduce or otherwise obscure the development over time of any residual bitter flavors and after taste in the nutritional composition. Suitable masking agents include natural and artificial sweeteners, sodium sources such as sodium chloride, and hydrocolloids such as guar gum, xanthan gum, carrageenan, gellan gum, and combinations thereof. The amount of masking agent used will often vary depending upon the particular masking agent selected, other ingredients in the formulation, and other formulation or product target variables.

Exemplary embodiments of the nutritional composition may also be substantially free of any optional or selected essential ingredient or feature described herein, provided that the remaining nutritional product still contains some of the required ingredients or features as described herein. In this context, and unless otherwise specified, the term “substantially free” means that the selected nutritional product contains less than a functional amount of the noted optional or selected essential ingredient, typically less than 1.0%, including less than 0.5%, less than 0.1%, and zero percent, by weight of such optional or selected essential ingredient.

Generally, the nutritional composition is formulated in a product form suitable for oral administration. Accordingly, the nutritional composition may take a wide variety of forms including, but not limited to, a liquid, a solid, a powder, a semi-solid, a semi-liquid, a nutritional supplement, and other forms of nutritional products known in the art.

In one exemplary embodiment, the nutritional composition may be a nutritional solid, nutritional liquid, nutritional semi-solid, nutritional semi-liquid, or nutritional powder. Examples of nutritional composition forms suitable for use in the exemplary methods include snack and meal replacement products, including those formulated as bars; sticks; cookies; breads, cakes, or other baked goods; frozen liquids; candy; breakfast cereals; powders, granulated solids, or other particulates; snack chips or bites; frozen or retorted entrees; and so forth. In certain exemplary embodiments, the nutritional composition can be in a form that falls between solid and liquid, such as puddings, yogurts, or gels. In certain exemplary embodiments, when the nutritional composition is a solid product, a serving thereof may be between about 25 grams and about 150 grams.

Exemplary liquid nutritional compositions include snack and meal replacement products, hot or cold beverages, carbonated or non-carbonated beverages, juices or other acidified beverages, milk or soy-based beverages, shakes, coffees, teas, and so forth. The liquid nutritional composition is most typically formulated as a suspension or an emulsion, but the liquid nutritional composition can also be formulated in any other suitable form such as a clear liquid, a substantially clear liquid, a liquid gel, and so forth. In certain exemplary embodiments, when the nutritional composition is a liquid, a serving thereof may be between about 115 milliliters and about 500 milliliters. In certain other exemplary embodiments, when the nutritional composition is a liquid, the serving is about 237 milliliters (˜8 fl. oz.). In other exemplary embodiments, when the nutritional composition is a liquid, the serving is between about 177 milliliters and about 414 milliliters (˜6 fl. oz. to ˜14 fl. oz.) or between about 207 milliliters and about 296 milliliters (˜7 fl. oz. to ˜10 fl. oz.).

In one exemplary embodiment, the nutritional composition is formulated as a clear liquid having a pH between 2 and 5, and also having no more than 0.5% fat by weight of the nutritional composition. The limited amount of fat contributes to the desired clarity of the nutritional composition. Typically, a liquid nutritional composition that is formulated to be clear, or at least substantially translucent, is substantially free of fat. As used herein “substantially free of fat” refers to a nutritional composition that contains less than 0.5% fat by weight of the composition, or less than 0.1% fat by weight of the composition. “Substantially free of fat” also may refer to a nutritional composition that contains no fat, i.e., zero fat. A liquid nutritional composition that is both clear and has a pH between 2 and 5 is also typically substantially free of fat. In certain exemplary embodiments, the pH of the nutritional composition may be between 2.5 and 4.6, including a pH between 3 and 3.5. In certain exemplary embodiments, when the nutritional composition is substantially free of fat, but has some amount of fat present, the fat may be present as a result of being inherently present in another ingredient (e.g., a source of protein) or may be present as a result of being added as one or more separate sources of fat.

The exemplary nutritional compositions may be prepared by any process or method (now known or known in the future) suitable for making a selected product form, such as a nutritional solid, a nutritional powder, or a nutritional liquid. Many such techniques may be known for any given product form, such as nutritional liquids or nutritional powders, and can readily be applied by one of ordinary skill in the art to the various exemplary embodiments described herein.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing selected oils (e.g., canola oil, corn oil) and then adding an emulsifier (e.g., soy lecithin), fat soluble vitamins, and a portion of the total protein (e.g., milk protein concentrate) with continued heat and agitation. The CHO-MN slurry is formed by adding with heated agitation to water: minerals (e.g., potassium citrate, magnesium phosphate, calcium carbonate), trace minerals and ultra trace minerals (e.g., TM/UTM premix), thickening or suspending agents (e.g., gellan gum, carrageenan), and HMB. The resulting CHO-MIN slurry is held for 10 minutes with continued heat and agitation before adding additional minerals (e.g., potassium chloride, magnesium carbonate, potassium iodide), and carbohydrates (e.g., fructooligosaccharides, sucrose). The PIW slurry is then formed by mixing with heat and agitation the remaining protein (e.g., sodium caseinate, soy protein isolate, whey protein concentrate) into water.

The resulting slurries are then blended together with heated agitation and the pH adjusted to a desired range, typically between about 6.6 and 7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range (if necessary), flavors are added, and water is added to achieve a desired total solid level. The composition is then aseptically packaged to form an aseptically packaged nutritional emulsion, or the composition is added to retort stable containers and then subjected to retort sterilization to form retort sterilized nutritional emulsions.

The manufacturing processes for the nutritional liquids may be carried out in ways other than those set forth herein without departing from the spirit and scope of the present general inventive concepts. The present embodiments are, therefore, to be considered in all respects illustrative and not restrictive with changes and equivalents intended to fall within the general inventive concepts.

A nutritional powder, such as a spray dried nutritional powder, may be prepared by any combination of known or otherwise effective techniques suitable for making and formulating a spray dried nutritional powder. The spray drying step may likewise include any spray drying technique that is known or otherwise suitable for use in the production of nutritional powders. Many different spray drying methods and techniques are known for use in the nutrition field, of which many are suitable for use in the manufacture of the spray dried nutritional powders herein.

One method of preparing an exemplary spray dried nutritional powder comprises forming and homogenizing an aqueous slurry or liquid comprising HMB, protein, carbohydrates, and fat, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder. In certain exemplary embodiments, the methods of manufacture utilize Ca-HMB. As previously discussed, the Ca-HMB is most typically formulated as a monohydrate salt.

Nutritional compositions according to the exemplary embodiments are useful for providing sole, primary, or supplemental sources of nutrition, as well as providing one or more of the benefits described herein such as maintaining intramuscular myoglobin levels, maintaining maximal aerobic capacity, and enhancing the oxidative capacity of muscle.

As previously discussed, myoglobin functions as a reserve supply of oxygen and facilitates the transport of oxygen within muscle tissue. Thus, maintaining intramuscular myoglobin levels is important for ensuring that adequate oxygen is supplied to the muscle tissue to support the oxidative capacity of the muscle. Maintaining or enhancing the oxidative capacity of muscle (through maintenance or an increase in intramuscular myoglobin levels) may maintain the maximal aerobic capacity of a subject in need thereof.

The exemplary methods described herein are provided to maintain intramuscular myoglobin levels, to maintain maximal aerobic capacity, to enhance the oxidative capacity of muscle, or to provide a combination of these benefits to a subject in need thereof. In one exemplary embodiment, the subject in need thereof is a human. In one exemplary embodiment, the subject in need thereof is an elderly human.

In one exemplary embodiment, the subject in need thereof has a low level of intramuscular myoglobin. In one exemplary embodiment, the subject in need thereof is hospitalized, immobilized, physically inactive, or on bed rest. The phrase “physically inactive,” as used herein, refers to a subject who does not engage in physical activity that exceeds 6 metabolic equivalent units (METs), does not engage in physical activity that exceeds 5 METs, or does not engage in physical activity that exceeds 3 METs. One MET is generally defined as the amount of energy expended while resting, often defined in terms of oxygen uptake as 3.5 mL O₂ per kg body weight×min. It has been shown that one of the side effects of bed rest is a reduction in maximal aerobic capacity (VO₂ max) (Capelli et al., Eur J Appl Physiol (2006); 98: 152-160).

In one exemplary embodiment, the subject in need thereof has a condition selected from joint disease, chronic obstructive pulmonary disorder, congestive heart failure, emphysema, cachexia, diabetes, sarcopenia, end stage renal disease, and asthma. Such conditions are known to affect muscle health, and may often be associated with a reduction in the oxidative capacity of muscle and a reduction in the maximal aerobic capacity of the subject.

Administration of an effective amount of β-hydroxy-β-methylbutyrate can maintain intramuscular myoglobin levels (by attenuating a decrease in serum myoglobin levels) in subjects in need thereof. Accordingly, the exemplary methods can be effective for maintaining intramuscular myoglobin levels, maintaining maximal aerobic capacity, and enhancing the oxidative capacity of muscle in a subject in need thereof.

As used herein, the phrase “maintaining intramuscular myoglobin levels” should be understood to include preserving intramuscular myoglobin levels or increasing intramuscular myoglobin levels. In this context, maintaining intramuscular myoglobin levels in a subject refers to retaining an amount of intramuscular myoglobin that corresponds to a measurement of the intramuscular myoglobin levels of the subject prior to initiating the exemplary methods disclosed herein, or a significant percentage thereof. Accordingly, in various exemplary embodiments, administering an effective amount of β-hydroxy-β-methylbutyrate (or a source thereof) results in maintaining 100% of the intramuscular myoglobin levels of the subject, or in other embodiments lesser amounts. For example, in certain exemplary embodiments, the method results in maintaining at least 50% of the intramuscular myoglobin levels of the subject. In certain other exemplary embodiments, the method results in maintaining at least 60% of the intramuscular myoglobin levels of the subject. In certain other exemplary embodiments, the method results in maintaining at least 70% of the intramuscular myoglobin levels of the subject. In certain other exemplary embodiments, the method results in maintaining at least 80% of the intramuscular myoglobin levels of the subject. In certain other exemplary embodiments, the method results in maintaining at least 90% of the intramuscular myoglobin levels of the subject. In certain other exemplary embodiments, the method results in maintaining at least 95% of the intramuscular myoglobin levels of the subject. In certain other exemplary embodiments, the method results in maintaining intramuscular myoglobin levels in amounts ranging between 50% and 100%, 50% and 80%, 50% and 90%, 60% and 80%, or 60% and 90%. In certain exemplary embodiments, the subject maintains 100% of their intramuscular myoglobin levels, or even increases their intramuscular myoglobin levels. Generally, when intramuscular myoglobin levels in a subject are “maintained” by more than 100%, this result is described herein as an increase in intramuscular myoglobin levels.

The intramuscular myoglobin levels may be determined by a wide variety of suitable methods (now known or known in the future). For example, in certain exemplary embodiments, the intramuscular myoglobin level may be determined by obtaining muscle tissue samples (e.g., muscle biopsy) and performing assays (e.g., ELISA, western blot, quantitative reverse transcription-polymerase chain reaction, RNase protection assay) to measure myoglobin levels in the muscle tissue. Moreover, muscle tissue samples can be obtained at different time points and subsequently assayed to calculate the change in the levels of myoglobin over time.

Additionally, serum myoglobin levels may provide an indication of intramuscular myoglobin levels. Serum myoglobin levels may be determined by a wide variety of suitable methods (now known or known in the future). For example, in certain exemplary embodiments, serum myoglobin levels may be determined by obtaining blood samples (e.g., serum, plasma) and performing assays (e.g., ELISA, western blot, quantitative reverse transcription-polymerase chain reaction, RNase protection assay) to measure myoglobin levels in the blood sample. Moreover, blood samples can be obtained at different time points and subsequently assayed to calculate the change in the levels of myoglobin over time. Muscle injury results in myoglobin being released from the muscle tissue into the bloodstream, and, thus, serum myoglobin is commonly used as a marker of muscle injury. Accordingly, a preservation of serum myoglobin levels may be indicative of a preservation of intramuscular myoglobin levels.

As used herein, the phrase “maintaining maximal aerobic capacity” should be understood to include preserving maximal aerobic capacity or increasing maximal aerobic capacity. In this context, maintaining maximal aerobic capacity in a subject refers to retaining an amount of the maximal aerobic capacity that corresponds to a measurement of the maximal aerobic capacity of the subject prior to initiating the exemplary methods disclosed herein, or a percentage thereof. Accordingly, in various exemplary embodiments, administering an effective amount of β-hydroxy-β-methylbutyrate (or a source thereof) results in maintaining 100% of the maximal aerobic capacity of the subject, or in other embodiments lesser amounts. For example, in certain exemplary embodiments, the method results in maintaining at least 75% of the maximal aerobic capacity of the subject. In certain other exemplary embodiments, the method results in maintaining at least 80% of the maximal aerobic capacity of the subject. In certain other exemplary embodiments, the method results in maintaining at least 85% of the maximal aerobic capacity of the subject. In certain other exemplary embodiments, the method results in maintaining at least 90% of the maximal aerobic capacity of the subject. In certain other exemplary embodiments, the method results in maintaining at least 95% of the maximal aerobic capacity of the subject. In certain other exemplary embodiments, the method results in maintaining maximal aerobic capacity in amounts ranging between 75% and 100%, 80% and 95%, or 85% and 90%. In certain exemplary embodiments, the subject maintains 100% of their maximal aerobic capacity, or even increases their maximal aerobic capacity. Generally, when the maximal aerobic capacity in a subject is “maintained” by more than 100%, this result is described herein as an increase in maximal aerobic capacity.

In one exemplary embodiment, maintaining intramuscular myoglobin levels is indicative of maintaining maximal aerobic capacity. For example, as discussed above, intramuscular myoglobin serves as an oxygen reserve in muscle tissue. Accordingly, maintaining or increasing intramuscular myoglobin levels may facilitate an increased amount of oxygen transport and utilization by the muscles, which can promote a maintenance or increase in maximal aerobic capacity.

The maximal aerobic capacity of a subject may be determined by a wide variety of suitable methods (now known or known in the future). For example, in certain exemplary embodiments, maximal aerobic capacity may be determined by a graded exercise test in which exercise intensity is progressively increased while analyzing the oxygen and carbon dioxide concentration of the inhaled and exhaled air. The maximal aerobic capacity is reached when oxygen consumption remains at steady state despite an increase in workload. In certain exemplary embodiments, indirect tests commonly used for predicting maximal aerobic capacity may be utilized, such as the Uth-Sørensen-Overgaard-Pedersen estimation. Moreover, the maximal aerobic capacity can be determined at different time points and subsequently compared to calculate the change in the maximal aerobic capacity over time.

As used herein, the phrase “enhancing the oxidative capacity of muscle” should be understood to include maintaining the oxidative capacity of muscle or increasing the oxidative capacity of muscle. In this context, maintaining the oxidative capacity of muscle in a subject refers to retaining an amount of the oxidative capacity of muscle that corresponds to a measurement of the oxidative capacity of muscle in the subject prior to initiating the exemplary methods disclosed herein, or a percentage thereof. Accordingly, in various exemplary embodiments, administering an effective amount of β-hydroxy-β-methylbutyrate (or a source thereof) results in maintaining 100% of the oxidative capacity of muscle in the subject, or in other embodiments lesser amounts. For example, in certain exemplary embodiments, the method results in maintaining at least 75% of the oxidative capacity of muscle in the subject. In certain other exemplary embodiments, the method results in maintaining at least 80% of the oxidative capacity of muscle in the subject. In certain other exemplary embodiments, the method results in maintaining at least 85% of the oxidative capacity of muscle in subject. In certain other exemplary embodiments, the method results in maintaining at least 90% of the oxidative capacity of muscle in the subject. In certain other exemplary embodiments, the method results in maintaining at least 95% of the oxidative capacity of muscle in the subject. In certain other exemplary embodiments, the method results in maintaining the oxidative capacity of muscle in a subject in amounts ranging between 75% and 100%, 80% and 95%, or 85% and 90%. In certain exemplary embodiments, the subject maintains 100% of their oxidative capacity of muscle, or even increases their oxidative capacity of muscle. Generally, when the oxidative capacity of muscle in a subject is “maintained” by more than 100%, this result is described herein as an increase (or enhancement) in the oxidative capacity of muscle in the subject.

The oxidative capacity of muscle in a subject may be determined by a wide variety of suitable methods (now known or known in the future). For example, in certain exemplary embodiments, the oxidative capacity of muscle may be determined by utilizing phosphorus magnetic resonance spectroscopy. In certain exemplary embodiments, the oxidative capacity of muscle may be determined by obtaining a muscle tissue sample (e.g., muscle biopsy) and determining the activity of citrate synthase (a mitochondrial enzyme and marker of muscle oxidative capacity) to measure the oxidative capacity of the muscle tissue. Moreover, muscle tissue samples can be obtained at different time points and subsequently assayed to calculate the change in the oxidative capacity of the muscle tissue samples between the time points.

In accordance with the exemplary methods described herein, an effective amount of β-hydroxy-β-methylbutyrate is administered to a subject in need thereof. The effective amount of β-hydroxy-β-methylbutyrate may be administered to the subject in one or multiple (e.g., two, three, four) doses or servings over a period of time. In one exemplary embodiment, an effective amount of β-hydroxy-β-methylbutyrate is administered to a subject in need thereof in two servings per day. In one exemplary embodiment, an effective amount of β-hydroxy-β-methylbutyrate is administered to a subject in need thereof within 2 hours of waking, within 2 hours of sleeping, or both within 2 hours of waking and within 2 hours of sleeping.

In accordance with certain exemplary methods disclosed herein, the effective amount of β-hydroxy-β-methylbutyrate can be administered to the subject in need thereof one or more times per day for a period of up to three weeks, or for a period of at least three weeks, to achieve the desired effect. For example, in certain exemplary embodiments, the effective amount of β-hydroxy-β-methylbutyrate can be administered to the subject in need thereof every day for at least three weeks, every day for at least four weeks, every day for at least eight weeks, every day for at least six months, or every day for a year or more. As another example, the effective amount of the β-hydroxy-β-methylbutyrate can be administered to the subject in need thereof twice a day for at least three weeks, twice a day for at least four weeks, twice a day for at least eight weeks, twice a day for at least six months, or twice a day for a year or more. In one exemplary embodiment, an effective amount of β-hydroxy-β-methylbutyrate can be administered to the subject in need thereof every day for up to one week, every day for up to two weeks, or every day for up to three weeks. Within the context of providing a dose or serving to the subject in need thereof, every day is intended to reflect a subject who has been instructed to be administered the β-hydroxy-β-methylbutyrate every day and who actually is administered the β-hydroxy-β-methylbutyrate for at least 70% (and in certain other exemplary embodiments at least 90%) of the days during the period of administration.

In certain exemplary embodiments, the β-hydroxy-β-methylbutyrate is acutely administered to the subject in need thereof. The phrases “acutely administered,” “acute administration,” and “acutely administering,” as used herein, refer to administering an effective amount of β-hydroxy-β-methylbutyrate to the subject in need thereof on a non-regular basis. Acute administration may be a single serving, or multiple servings, administered over a relatively short time period, such as up to three weeks, including one day, two days, three days, five days, one week, ten days, two weeks, or three weeks.

In certain exemplary embodiments, the β-hydroxy-β-methylbutyrate is chronically administered to the subject in need thereof. The phrases “chronically administered,” “chronic administration,” and “chronically administering,” as used herein, refers to regular administration which is provided indefinitely, or to regular administration for a significant period of time. For example, in certain exemplary embodiments, chronic administration can include regular administration for at least three weeks, regular administration for at least one month, regular administration for at least 6 weeks, regular administration for at least two months, regular administration for at least 3 months, regular administration for at least 4 months, regular administration for at least 5 months, regular administration for at least 6 months, or regular administration for at least 9 months. In other exemplary embodiments, chronic administration refers to regular administration for at least 1 year, regular administration for at least 1.5 years, regular administration for at least 2 years, or regular administration for more than 2 years. “Regular administration,” as used herein, refers to administration according to a schedule whereby the subject in need thereof will receive the β-hydroxy-β-methylbutyrate at regular intervals.

As used herein, “regular intervals” refers to administration in a repeating, periodic fashion where the time between administrations is approximately (or intended to be approximately) the same. In various exemplary embodiments, administration at regular intervals includes daily administration or weekly administration. In other exemplary embodiments, administration at regular intervals includes administration 1-2 times per week, administration 1-3 times per week, administration 2-3 times per week, administration 1-4 times per week, administration 1-5 times per week, administration 2-5 times per week, administration 3-5 times per week, administration 1-6 times per week, administration 1-7 times per week, administration 2-6 times per week, administration 2-7 times per week, administration 1-2 times per day, administration 1-3 times per day, administration 1-4 times per day, administration 2-3 times per day, administration 2-4 times per day, administration 3-4 times per day, administration 2-5 times per day, administration 3-5 times per day, administration 4-5 times per day, or administration more than 5 times per day.

In terms of measuring “maintenance of intramuscular myoglobin levels,” “maintenance of maximal aerobic capacity,” or “enhancement of the oxidative capacity of muscle,” a first measurement of a parameter (i.e., intramuscular (or serum) myoglobin levels, maximal aerobic capacity, oxidative capacity of muscle) is performed prior to initiating the methods disclosed herein. In certain exemplary embodiments, the first measurement is performed a week (e.g., 1-7 days or 7 days) before initiation of the exemplary methods. Next, a second measurement of the parameter of the subject is performed at some time point after initiating the exemplary methods, and the second measurement is compared to the first measurement. Notably, the comparison of the second measurement to the first measurement may not show immediate results using the aforementioned measurement techniques. The resulting effect may take days, weeks, or months of regular administration of β-hydroxy-β-methylbutyrate (or nutritional compositions containing β-hydroxy-β-methylbutyrate) according to the dosages and in the intervals previously described above to obtain the stated benefits described herein.

In certain exemplary embodiments, measuring the maintenance or enhancement of a parameter (i.e., intramuscular (or serum) myoglobin levels, maximal aerobic capacity, oxidative capacity of muscle) may require an amount of time between the first measurement and the second measurement, such as two weeks, one month, two months, six months, or longer. In certain exemplary embodiments, for the purposes of determining the effects of administering β-hydroxy-β-methylbutyrate as disclosed herein, a 3-12 month test period of regular administration of β-hydroxy-β-methylbutyrate may be used. In certain other exemplary embodiments, for the purposes of determining the effects of administering β-hydroxy-β-methylbutyrate, a 2 week to 3 month test period of regular administration of β-hydroxy-β-methylbutyrate may be used.

### EXAMPLES

The following examples illustrate certain exemplary embodiments of the methods and nutritional compositions disclosed herein. The examples are given solely for the purpose of illustration and are not to be construed as limitations of the general inventive concepts, as many variations thereof are possible without departing from the spirit and scope of the general inventive concepts.

### Example 1

An exemplary nutritional composition in the form of a nutritional liquid emulsion suitable for use in the methods disclosed herein is described in Example 1, with the specific ingredients provided immediately thereafter.

| Example 1 | UNIT | per 8 fl oz |
|---|---|---|
| Energy EU | kcal | 350 |
| Nutrient Density | kcal/ml | 1.5 |
| Protein | % kcal | 15 |
| Carbohydrate | % kcal | 57 |
| Fat | % kcal | 28 |
| Osmolality | mOsm/kg | 850 |
| | H₂O | |
| Protein | g | 13 |
| Ca-HMB | g | 1.5 |
| Total Fat | g | 20 |
| Polyunsaturated Fat | g | 4 |
| Monounsaturated Fat | g | 6 |
| Total Carbohydrate | g | 51 |
| Fructooligosaccharide | g | 3 |
| Sugar | g | 20 |
| VITAMINS | | |
| Vitamin A | IU | 1250 |
| Vitamin C | mg | 36 |
| Vitamin D | IU | 160 |
| Vitamin E | IU | 9 |
| Vitamin K | mcg | 20 |
| Folic Acid | mcg | 100 |
| Vitamin B₁ | mg | 0.38 |
| Vitamin B₂ | mg | 0.43 |
| Vitamin B₆ | mg | 0.5 |
| Vitamin B₁₂ | mcg | 1.5 |
| Niacin | mg | 5 |
| Pantothenic Acid | mg | 2.5 |
| Biotin | mcg | 75 |
| Choline | mg | 83 |
| MINERALS | | |
| Sodium | mg | 240 |
| Potassium | mg | 840 |
| Chloride | mg | 140 |
| Calcium | mg | 350 |
| Phosphorus | mg | 350 |
| Magnesium | mg | 100 |
| Iron | mg | 4.5 |
| Zinc | mg | 3.8 |
| Manganese | mg | 1.2 |
| Copper | mg | 0.50 |
| Iodine | mcg | 38 |
| Selenium | mcg | 21 |
| Chromium | mcg | 30 |
| Molybdenum | mcg | 45 |

The exemplary nutritional composition described in Example 1 includes Water, Corn Maltodextrin, Sugar (Sucrose), Canola Oil, Sodium Caseinate, Milk Protein Concentrate, Corn Oil, Short-Chain Fructooligosaccharides, Soy Protein Isolate, Potassium Citrate, Calcium Beta-Hydroxy-Beta-Methylbutyrate (Ca-HMB), and less than 0.5% of the following: Whey Protein Concentrate, Natural & Artificial Flavors, Magnesium Phosphate, Soy Lecithin, Sodium Phosphate, Potassium Phosphate, Choline Chloride, Ascorbic Acid, Calcium Carbonate, Potassium Chloride, L-Carnitine, Carrageenan, Ferrous Sulfate, dl-Alpha-Tocopheryl Acetate, Zinc Sulfate, Gellan Gum, Niacinamide, Manganese Sulfate, Calcium Pantothenate, Cupric Sulfate, Vitamin A Palmitate, Thiamine Chloride Hydrochloride, Pyridoxine Hydrochloride, Riboflavin, Folic Acid, Chromium Chloride, Biotin, Sodium Molybdate, Sodium Selenate, Potassium Iodide, Phylloquinone, Vitamin D₃, and Cyanocobalamin.

### Example 2

An exemplary nutritional composition in the form of a clear liquid suitable for use in the methods disclosed herein is described in Example 2. All ingredient amounts listed in Example 2 are listed as kilogram per 1000 kg batch of product, unless otherwise indicated. The exemplary nutritional composition is substantially free of fat and has a pH in the range of about 3 to about 3.5. Assuming a density of 1.05 g/mL and a serving size of about 296 mL (about 10 fl. oz.), the exemplary nutritional composition described in this example has about 9 grams of protein per serving (or about 0.0304 g/mL), about 35 grams of carbohydrate per serving (or about 0.118 g/mL), 0 grams of fat per serving, and an energy content of 180 kcal per serving (or about 0.61 kcal/mL). Additionally, the exemplary nutritional composition includes about 1.5 grams of Ca-HMB per serving.

EXAMPLE 2

| INGREDIENTS | Amount (kg/1000 kg) |
|---|---|
| Water | Quantity Sufficient |
| Sucrose | 50.7 |
| Corn syrup solids | 61.3 |
| Acidified Whey Protein Isolate | 35.7 |
| Citric Acid | 2.00 |
| Flavoring | 2.00 |
| Ca-HMB | 4.83 |
| Ascorbic Acid | 0.535 |
| Liquid Sucralose (25%) | 0.275 |
| Ultra Trace Mineral/Trace Mineral Premix | 0.230 |
| Vitamin Premix¹ | 0.219 |
| Acesulfame Potassium | 0.110 |
| Antifoam processing aid (non-silicone) | 0.060 |
| Coloring | 0.0589 |
| Natural and Artificial Peach Flavor | 2.0 |
| Folic Acid | 0.0013 |
| Potassium Iodide | 0.000204 |

¹Vitamm premix includes one or more of the following: dl-Alpha-Tocopheryl Acetate, Vitamin A Palmitate, Phylloquinone, Vitamin D₃, Niacinamide, d-Calcium Pantothenate, Thiamine Chloride Hydrochloride, Pyridoxine Hydrochloride, Riboflavin, Folic Acid, Biotin, Cyanocobalamin, etc.

### Example 3

Example 3 describes a clinical study that was conducted to evaluate the effects of 10 days of bed rest on healthy elderly subjects. The exemplary methods are based, at least in part, on inventors' discovery that serum myoglobin levels change by statistically significant amounts in 18 healthy elderly subjects undergoing 10 days of bed rest. The exemplary methods are also based, at least in part, on inventors' discovery that intervention with HMB altered the change in serum myoglobin levels.

Subjects

The following inclusion criteria were verified at screening: male or female ≧60 to ≦79 years of age; body mass index (BMI)>20 but <35; ambulatory with a Short Performance Physical Battery (SPPB) score of >9 (fully functional with no mobility limitations); and compliance with prescribed activity level. Exclusion criteria ruled out subjects who had undergone recent major surgery, had active malignancy (excepting basal or squamous cell skin carcinoma or carcinoma in situ of the uterine cervix); history of Deep Vein Thrombosis (DVT) or other hypercoagulation disorders; refractory anemia; history of diabetes or fasting blood glucose value >126 mg/dL; presence of partial or full artificial limb; kidney disease or serum creatinine >1.4 mg/dL; evidence of cardiovascular disease assessed during resting or exercise EKG; untreated hypothyroidism; liver disease; chronic or acute GI disease; uncontrolled severe diarrhea, nausea or vomiting; were actively pursuing weight loss; were enrolled in other clinical trials; could not refrain from smoking over the bed rest study period; or could not discontinue anticoagulant therapy over bed rest period. Potential subjects were also excluded if they were taking any medications known to affect protein metabolism (e.g., progestational agents, steroids, growth hormone, dronabinol, marijuana, HMB, free amino acid supplements, dietary supplements to aid weight loss).

The 24 healthy subjects initially involved in the study were randomized into two groups. Subjects in the treatment group received two β-hydroxy-β-methylbutyrate (HMB) sachets containing 1.5 grams of Ca-HMB (TSI, Salt Lake City, Utah), 4 grams of maltodextrin, and 200 milligrams of calcium with additional sweetener and flavoring agents. Subjects in the control group received two control sachets that were identical to the HMB sachets with the exclusion Ca-HMB. This study was a double-blinded study. Neither the investigators nor the subjects were informed of the identity of any of the study products during the clinical portion of the study. Subjects were instructed to consume a sachet twice daily by mixing a sachet into a non-caloric, non-caffeinated, non-carbonated, non-milk-based beverage of their choice. Treatment with HMB or Control was initiated 5 days prior to bed rest and was continued until the end of the 10 day bed rest period.

For diet stabilization over the pre-bed rest and bed rest period, subjects were fed a metabolically controlled diet providing the RDA for protein intake (0.8 g protein/kg body weight per day). Total calorie needs were estimated using the Harris-Benedict equation for resting energy expenditure according to the following equation: For women=[655+(9.56×body weight in kg)+(1.85×height in cm)−(4.68×age in years)]×AF, and, For men=[66+(13.7×body weight in kg)+(5×height in cm)−(608×age in years]×AF, where AF=activity factor of 1.6 for the ambulatory period and 1.35 for the bed rest period. Given the total calorie and protein intakes, the remainder of the diet was manipulated to keep the non-protein calories at about 60% from carbohydrates and 40% from fat. Water was provided ad libitum.

After a diet stabilization of 5 days (ambulatory period), subjects remained in bed continuously for 10 days. While confined to bed rest, subjects were allowed to use the bedside commode for urination or were taken in a wheelchair for toileting. Subjects were given the option of taking a sponge bath or showering in a wheelchair. Prophylactic measures were taken to detect and prevent deep vein thrombosis including a blood D-dimer test followed by an ultrasound examination if D-dimer test was positive, passive range of motion exercise during bed rest, the use of TED hose and SCD over the bed rest period. Subjects were offered medication to help mitigate reflux problems associated with being supine. Subjects were constantly monitored by nursing staff and received a daily physical examination by the study physician.

Fasted blood samples were collected from subjects on Day 1 of bed rest and at the end of bed rest for measurement of biomarkers, including myoglobin.

Subjects were exited from the study if they permanently discontinued product during the pre-bed rest period (Day 1 to Day 5), or if they discontinued product during the bed rest period and had completed less than 8 days of bed rest. Subjects with a positive D-dimer test or ultrasound for deep vein thrombosis (DVT) diagnosis were also exited from the study.

A subject's outcome data were classified as unevaluable for the analysis if one or more of the following events occurred: A. Subject received wrong product, contrary to the randomization scheme; B. Subject received excluded concomitant treatment defined as medications or dietary supplements that affect weight or metabolism (e.g., progestational agents, steroids, growth hormone, dronabinol, marijuana, HMB, free amino acid supplements, dietary supplements to aid weight loss, and fish oil supplements); and C. Subject had <67% of total study product consumption at Final Visit/Exit as determined by product consumption records.

The final analytic sample size was n=18 subjects, n=8 in the control group (n=1 male, n=7 female) and n=10 (n=2 male, n=8 female) in the experimental HMB group.

Body Composition

Body weight was measured at baseline and after bed rest to the nearest 0.1 kilogram on an Ohaus scale (Ohaus Corporation, model 15S, Florham Park, N.J.). Nude body weight was calculated as total body weight minus hospital robe weight. Body height was measured to the nearest 0.1 cm without shoes using a stadiometer. Body mass index (BMI) was calculated as weight/height² (kg/m²). Measurements of body composition were conducted prior to and at the end of the 10 day bed rest period. DXA (Hologic Delphi W running QDR System Software Version 11.2) was used to estimate total and lower extremity LBM using a standard protocol (Kortbein, P. et al, JAMA (2007); 297 (16): 1772-4).

Biomarker Analysis

Rules Based Medicine (Myriad RBM, Inc., Austin, Tex.) data generated from the RBM Human DiscoveryMAP v1.0 consists of n=187 biomarkers measured in serum collected at two time points, pre-bed rest and post-bed rest from 18 elderly subjects. The distribution of each marker was evaluated. Each marker has a least detectable dose (LDD) value, defined as the mean+3 standard deviations (SD) of 20 blank samples. For any subject whose marker result was <LDD, the LDD value as provided by RBM was imputed. If the marker result was >LDD, the original result was used. Any marker in which >30% of all subject's results were imputed was excluded from further statistical analyses. Of the initial n=187 RBM markers, n=63 markers from the RBM dataset were excluded from further analyses, leaving n=124 markers for evaluation.

Statistical Evaluation

Changes in RBM Biomarkers

The Control group was examined to determine the biomarkers that changed over bed rest. Individual univariate dependent t-tests were performed on each of the 124 markers. There were a total of 8 participants who had matched data over bed rest within the Control group. From the initial univariate analysis, 13 biomarkers, including myoglobin, showed a statistically significant change over bed rest, with an unadjusted p-value less than 0.05.

Significance of ANCOVA tests

In order to assess the changes in the markers over bed rest that may be mediated by HMB intervention, individual univariate ANCOVA analyses were subsequently performed on each of the 13 (unadjusted) significant markers from the multiple dependent t-tests. Using a Bonferonni adjusted p-value of 0.0038 (0.05/13), 10 markers, including myoglobin, were significant. This result indicates that the serum myoglobin levels showed a statistical difference after bed rest between the Control and HMB groups while controlling for existing differences at baseline (pre-bed rest).

Results

Body Composition

There was no significant change in total body weight over the bed rest. Total lean mass was measured by Dual Energy X-Ray Absorptiometry (DXA). The Control group lost an average of −2.05±0.66 kg total lean mass (p=0.02, paired t test), whereas the HMB group lost an average of −0.17±0.19 kg total lean mass (p=0.42, paired t-test). Comparison of the change value in total lean mass over the bed rest period between treatment groups was statistically significant (p=0.02, ANOVA). Similar results were obtained for leg lean mass with the HMB group showing an average loss of −0.08±0.17 kg (p=0.65, paired t-test) versus the Control (−1.01±0.35 kg, p=0.02, paired t-test). There was a statistically significant difference in the change value of leg lean mass over the bed rest period between treatment groups (p=0.02, ANOVA). Accordingly, the data indicate that intervention with HMB improves retention of muscle mass during bed rest.

Myoglobin Levels

Myoglobin is a protein that serves as a reserve supply of oxygen and facilitates the transport of oxygen within muscle tissue. Serum myoglobin levels are commonly used as a marker of muscle damage, but serum myoglobin levels are sensitive to a number of physiological parameters, such as muscle membrane integrity and intramuscular myoglobin concentrations. As shown in Table 1, there was an average decrease of 31.6 ng/ml (or a 38% decrease) in serum myoglobin levels in 8 control subjects after 10 days of bed rest. In contrast, the average decrease in serum myoglobin levels in blood from 10 subjects treated with HMB was much less. As shown in Table 1, there was an as an average decrease of 10.2 ng/ml (or a 19% decrease) in blood levels of myoglobin in the HMB treated subjects. These results show that HMB reduces or attenuates the decrease in blood levels of myoglobin that occurs in untreated control subjects during prolonged bed rest.

As discussed above, treatment with HMB was associated with improved muscle mass retention during bed rest. This result suggests that treatment with HMB does not negatively impact muscle membrane integrity, which indicates that relative myoglobin efflux is unchanged. Accordingly, the study data supports a finding that treatment of subjects on bed rest with HMB results in a preservation of serum myoglobin, which is indicative of a preservation or increase of intramuscular myoglobin. This maintenance or increase of intramuscular myoglobin promotes oxygen transport and oxidative metabolism in the muscles of the subject. Moreover, the promotion of oxygen transport and oxidative metabolism through the maintenance or increase of intramuscular myoglobin may maintain or enhance the overall maximal aerobic capacity of the subject.

TABLE 1

| CONTROL | Myoglobin (ng/ml) | | | | | |
|---|---|---|---|---|---|---|
| (n = 8) | Pre-bed rest | | Post-bed rest | | | |
| | Mean | Stdev | Mean | Stdev | Change | % Change |
| | 72.4 | 48.6 | 40.8 | 20.6 | −31.6 ± 52.8 | −38 ± 8 |
| HMB | Myoglobin (ng/mL) | | | | | |
| (n = 10) | Pre-bed rest | | Post-bed rest | | | |
| | Mean | Stdev | Mean | Stdev | Change | % Change |
| | 49.3 | 5 | 39.1 | 4 | −10.2 ± 6.4 | −19 ± 6 |

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use.

While the present disclosure illustrates the general inventive concepts by describing various exemplary embodiments thereof, and while the embodiments may be described in considerable detail, the exemplary embodiments are not intended to restrict or in any way limit the scope of the general inventive concepts, including the appended claims, to such detail. Additional advantages and modifications will be readily apparent to those skilled in the art. Therefore, the general inventive concepts, in their broader aspects, are not limited to the specific details, the representative compositions and methods, or the illustrative examples shown and described. Accordingly, departures may be made from such details without departing from the spirit or scope of the general inventive concepts.

What is claimed is:

1. A method of maintaining intramuscular myoglobin levels in a subject in need thereof during a period of physical inactivity, the method comprising administering an effective amount of β-hydroxy-β-methylbutyrate to the subject in need thereof.

2. The method according to claim 1, wherein the β-hydroxy-β-methylbutyrate is administered orally.

3. The method according to claim 1, wherein about 0.1 g/day to about 10 g/day of β-hydroxy-β-methylbutyrate is administered to the subject in need thereof.

4. The method according to claim 1, wherein the β-hydroxy-β-methylbutyrate is administered as part of a nutritional composition.

5. The method according to claim 4, wherein the nutritional composition comprises at least one of a source of protein, a source of carbohydrate, and a source of fat.

6. The method according to claim 1, wherein the subject in need thereof is an elderly human.

7. The method according to claim 1, wherein the subject in need thereof is hospitalized, immobilized, physically inactive, or on bed rest.

8. A method of maintaining maximal aerobic capacity of a subject in need thereof, the method comprising:
administering an effective amount of β-hydroxy-β-methylbutyrate to the subject in need thereof;
wherein administration of the β-hydroxy-β-methylbutyrate increases intramuscular myoglobin levels, and thereby maintains the maximal aerobic capacity of the subject.

9. The method according to claim 8, wherein the β-hydroxy-β-methylbutyrate is administered orally.

10. The method according to claim 8, wherein about 0.1 g/day to about 10 g/day of β-hydroxy-β-methylbutyrate is administered to the subject in need thereof.

11. The method according to claim 8, wherein the β-hydroxy-β-methylbutyrate is administered as part of a nutritional composition.

12. The method according to claim 11, wherein the nutritional composition comprises at least one of a source of protein, a source of carbohydrate, and a source of fat.

13. The method according to claim 8, wherein the subject in need thereof is an elderly human.

14. The method according to claim 8, wherein the subject in need thereof is hospitalized, immobilized, physically inactive, or on bed rest.

15. The method according to claim 8, wherein the subject in need thereof has a condition selected from joint disease, chronic obstructive pulmonary disorder, congestive heart failure, emphysema, and asthma.

16. A method of enhancing the oxidative capacity of muscle in a subject in need thereof, the method comprising:
administering an effective amount of β-hydroxy-β-methylbutyrate to the subject in need thereof;
wherein administration of the β-hydroxy-β-methylbutyrate increases intramuscular myoglobin levels, and thereby enhances the oxidative capacity of muscle in the subject.

17. The method according to claim 16, wherein the β-hydroxy-β-methylbutyrate is administered orally.

18. The method according to claim 16, wherein about 0.1 g/day to about 10 g/day of β-hydroxy-β-methylbutyrate is administered to the subject in need thereof.

19. The method according to claim 16, wherein the β-hydroxy-β-methylbutyrate is administered as part of a nutritional composition.

20. The method according to claim 19, wherein the nutritional composition comprises at least one of a source of protein, a source of carbohydrate, and a source of fat.

21. The method according to claim 16, wherein the subject in need thereof is an elderly human.

22. The method according to claim 16, wherein the subject in need thereof is hospitalized, immobilized, physically inactive, or on bed rest.

23. The method according to claim 16, wherein the subject in need thereof has a condition selected from joint disease, chronic obstructive pulmonary disorder, congestive heart failure, emphysema, and asthma.
